(19) United States
(12) Patent Application Publication
(10) Pub. No.: US 2025/0268559 A1
(43) Pub. Date: Aug. 28, 2025

(54) NON-INVASIVE RESONANT DETECTION OF PRESSURE

(71) Applicant: Keith Michael Nord, Jackson, TN (US)

(72) Inventor: Keith Michael Nord, Jackson, TN (US)

(21) Appl. No.: 18/588,868

(22) Filed: Feb. 27, 2024

(51) Int. Cl. A61B 8/08; A61B 8/00

(52) U.S. Cl. CPC A61B 8/08; A61B 8/4227; A61B 8/4483; A61B 8/5223

(57) ABSTRACT

A device and method of measuring internal pressures without direct contact with the internal liquid by using external sound wave (vibration) emitters to generate a range of low frequency vibrations or sound waves to induce the natural resonance points at multiple frequencies within a body part or fluid container where the internal pressure measurement is desired, focusing on muscle fascial compartment pressure measurement. A separate microphone receiver or receivers detect the different resonant frequencies of the container or body part while it is being vibrated by the emitters, and estimates the pressure inside the container without direct sampling through mathematical computations and comparison of the resonant values to values obtained previously from laboratory control tests with known pressure values or healthy contralateral limbs.

### FIELD OF THE INVENTION

The present invention relates generally to a medical device and methods, more particularly, to a device that generates low to ultra-low sound wave vibration frequencies to induce resonance of body tissues or fluids at several different resonant frequencies to calculate and/or estimate the pressure of a container, such as muscle fascial compartments, eyeballs, or spinal fluid in the spinal canal or cranial cavity, without the need for piercing the tissue or container with a needle or similar style measuring device. This device focuses particularly on measurement of muscle fascial compartment pressure. The device could similarly be used outside of the medical field in sensitive applications that prohibit measurement of pressure by direct contact with the fluid or material, such as measurement of toxic or caustic liquids.

### BACKGROUND OF THE INVENTION

Accurate and reliable pressure measurement is a requirement for the safe operation of most industrial processes. Pressure measurement is particularly important in the medical field to help diagnose and treat a variety of diseases and pathological conditions. Common medical conditions in which pressure measurements are crucial are compartment syndrome in the arms and legs, glaucoma of the eyes, increased intracranial pressure (ICP), and hypertension, The most common pressure sensors employ a Bourdon tube, diaphragm, bellows, force balance, or variable capacitance arrangement. The most common methods of pressure measurement in medicine are as follows.

Compartment Pressure Measurement: Muscles in the arms and legs are arranged into groups called compartments, wrapped in tough protective organic sheet-like structure called fascia. The pressure inside a compartment may increase after crushing injury, bone fracture, metabolic conditions or other injuries. Persistently elevated pressure beyond a critical threshold leads to a cascade of further injury called compartment syndrome, which is an emergent medical condition that requires immediate surgery to prevent permanent muscle death, nerve damage, and life-long disability. If a physician determines there are concerns for compartment syndrome, a measurement is typically obtained from each compartment to confirm the diagnosis. To measure compartment pressure, a large bore needle is inserted through the skin and fascia into the muscle compartment by a physician, and a small amount of fluid is injected to establish direct contact of fluid at pressure with an external pressure gauge to measure pressure. This is a painful, invasive procedure that may risk damage to internal structures, bleeding or infection. This method cannot safely be done in all regions of the body as it relies on inserting a large needle into the tissue to establish direct contact with the fluid for measurement by an external pressure gauge. A needle must be inserted into each compartment, often at three or four locations depending on the body part, causing significant pain to the patient. Normal muscle compartment pressure is 0-8 mmHg. Pressures within 30 mmHg of the diastolic blood pressure indicate compartment syndrome.

Cerebrospinal Fluid (CSF) Pressure Measurement: Increased fluid pressure in the skull or spinal canal can be caused by brain swelling or hydrocephalus, and is associated with seizures, vomiting, nerve dysfunction, loss of consciousness, coma, altered breathing, or even death. CSF pressure may be measured by lumbar puncture or by directly inserting a tube or bolt through the skull into the space between the skull and the brain. To measure the CSF pressure by lumbar puncture, a cannulated needle is inserted through the skin, fascia, and dura to enter the arachnoid space, allowing spinal fluid to flow into the needle at the same pressure as the internal space, allowing direct measurement of pressure with an external gauge. This is the same underlying concept as a traditional compartment pressure measurement. This requires an invasive and painful procedure that risks damage to internal structures, bleeding, and possible infection.

Eye Pressure (Intraocular) Measurement: Tonometry. Ocular hypertension (IOP), is often caused by failure of fluid to drain from the eye. Chronically increased IOP can cause optic nerve damage or blindness. To measure intraocular pressure, the eyeball's outer layer, the cornea, is struck with a tiny probe or blast of air to cause a minor deflection of the outer wall (cornea) temporarily, which allows indirect measurement of pressure by measuring the extent of deflection of the container and the amount of force required to cause the deflection. A healthy intraocular pressure is between 10 mmHg and 20 mmHg. This method only works in body parts that can directly contacted or struck and measured. Internal structures such as muscle facial compartments and the spinal canal cannot be measured this way because they cannot easily be directly struck or measured because they are covered by layers of skin and adipose tissue.

Blood Pressure Measurement: Sphygmomanometer. Elevated blood pressure can be caused by stress, arterial disease, kidney disease, drugs, diet and other unhealthy lifestyle choices. Blood pressure may be checked by direct sampling of the blood by an atrial catheter, or non-invasively by using a sphygmomanometer (blood pressure cuff). With a sphygmomanometer, a blood vessel or artery is completely occluded temporarily by applying a pressure much greater than the blood pressure, then slowly releasing the pressure until blood begins to flow through the artery again. The pressure at which point the blood begins to flow initially, the systolic pressure, causes turbulent flow and vibrations that can be detected by a stethoscope or ultrasound probe. The pressure at which no more turbulent flow is detected is the diastolic pressure. This method does not work for muscle fascial compartments or sensitive internal structures such as the spinal canal as there is no flow turbulence to measure and application of large amounts of pressure in these regions is neither feasible nor safe.

Resonance is when a vibrating object causes another object to vibrate at a higher amplitude than itself. Resonance happens when the frequency of the initial object's vibration matches the resonant frequency or natural frequency of the second object. The second object vibrates or oscillates at a higher amplitude as a result of resonance. Objects and systems naturally vibrate at a definitive frequency, called the resonant frequency or natural frequency. The resonant frequency is determined by the size and shape of the object, along with the material it is made from and its elasticity. When a guitar string is plucked, the resonating vibrations cause sound waves in the surrounding air. The frequencies of sound waves produced by guitar strings are the resonant frequencies of the string. If a guitar string is subjected to vibrations from an emitter that is generating waves at the natural frequency of the string, an amplified sound wave will be detectable by a receiver (the ear). If physical properties of the string are known, such as mass and length, the tensile force (tension) in the string can be calculated. The tensile force (tension) of the guitar string can be calculated using the formula:

$${f}_{n} = ( \frac{n}{2} ) {( \frac{T}{LM} )}^{1 / 2}$$

where L is string length, M is mass, T is tension, f is the frequency, and n is the N-th harmonic. The lowest frequency for resonance is called the fundamental frequency. All frequencies higher than the fundamental frequency that induce resonance are called overtones. Real world objects have many natural frequencies, and it is often a critical design necessity to identify these natural resonant frequencies to prevent failure due to excessive vibrations.

The human body has been found to have many natural frequencies, as well as different natural frequencies for each body part. The human whole-body fundamental resonant frequency is estimated to be between 5-10 Hz, depending on the magnitude of the vibration used. The lower arm has been estimated to have resonant frequency 16-30 Hz. The hand has resonant frequency between 30-50 Hz. The legs have been estimated to have a resonant frequency between 2-20 Hz depending on the knee position and body composition. The eyeball and intraocular structures have resonant frequencies between 20-90 Hz. The head has a resonant frequency of 20-30 Hz in an axial mode. The spinal column has a resonant frequency of 10-12 Hz in axial mode.

Accordingly, there is an established need for a non-invasive device that could measure the pressure of compartments in the body without the need for inserting large needles and catheters that can cause infection, pain, and damage to nearby structures. Such a device would be useful outside of the medical field as well when there is a need to measure pressure without directly contacting the liquid in question.

### SUMMARY OF THE INVENTION

The present invention is directed to a low frequency resonant pressure detection device and method provided for non-invasive measurement of pressure in a container or body part, with a focus on the measurement of pressure in muscle compartments of the arms or legs. The device consisting of an emitter or emitters that generate a range of low frequency vibrations or sound waves at varying amplitudes, that at certain frequencies, will induce the natural resonant frequencies in a container or body part where the pressure is to be measured. Every physical object has several natural resonant frequencies that can be measured. These natural frequencies will change based on the physical properties and stresses on container or body part, such as elevated pressure. The device will also have one or multiple microphone receivers that will be listening to detect and record the induced natural resonant frequencies. These frequencies values can then be used in mathematical calculations, comparing them to previously obtained data points and average values from laboratory and cadaver experiments to generate an estimation of the internal compartment pressure.

A conceptual example of this concept is a guitar string. A string of known density can be vibrated at a resonant frequency to generate a sound wave that propagates through the air. A sensor can detect this frequency and it is then possible to calculate the tension in the string through mathematical equations. Following the principles of calculus, a line of guitar strings side-by-side is similar to a sheet of muscle fascia. The tension of the sheet can be calculated by listening for the resonant frequency points. A sheet of fascia rolled into a cylinder is representative of a muscle fascial compartment. Calculating the tension in the individual strands of fascia and then extrapolating that to the surface area of the compartment can allow indirect calculation of the compartment pressure. It may be difficult to isolate an individual compartment, but the average measurement of all the compartments in the leg compared to known normal values obtained from the opposite limb can be used to detect elevated pressures that may indicate compartment syndrome and the potential need for more invasive testing or surgical treatment.

In another aspect, the device may be incorporated into a blood-pressure cuff type sleeve to allow easy application of the emitters and receivers to an arm or leg.

In another aspect, the device may be incorporated or housed in a solid or flexible housing component with a central circular or ovoid opening that an arm or leg may be placed inside of temporarily during pressure measurements.

In a second implementation of the invention, a method of pressure measurement and comparison by using the device on the contralateral, uninjured limb of a patient to obtain baseline natural resonant frequency and pressure values. The device would then be moved to the injured limb where compartment syndrome and elevated pressures are a concern. Measurements would then be taken on the injured limb and used in mathematical calculations and compared to the previously obtained values. An increase in measured values could indicate elevated pressures and the need for more invasive traditional testing, or surgery. This method is of particular value as it allows comparison of two nearly identical, isomeric limbs with similar body composition, with potentially the only difference being the current injury causing elevated pressures, minimizing potential confounders and increasing accuracy.

In a third implementation of the invention, a method for diagnosing compartment pressure by measuring the pressure multiple times and detecting a change in the natural resonant frequency points over time. Because the device is non-invasive, it could be used multiples times to obtain serial measurements of an injured extremity to ensure compartment syndrome does not develop over time. This would also potentially allow measurement of compartment pressure by nursing staff. Currently, compartment pressure measurements are done only by medical doctors as it is an invasive procedure. Existing invasive pressure measurement methods could be used to confirm the elevated pressure after being alerted to increasing pressure over time with the non-invasive device.

In another aspect, the device could be used on the eyes to measure intraocular pressure. The device could be used on the contralateral, normal eyeball, to obtain baseline resonant values that could then be compared to the pathologic (abnormal) eyeball. An increase or decrease in natural resonant values could be compared to the normal values or used in mathematical calculations for medical diagnosis or to signal the need for more traditional testing.

In another aspect, the device could be used on the spinal cord or head to measure the intracranial pressure or cerebrospinal fluid pressure. The device could be applied to the lumbar region or to the head to measure the pressure in these locations. Baseline resonant frequency values would be obtained from previously performed studies on cadavers or patients with known elevated and normal pressures to help increase pressure measurement accuracy.

In another aspect, the device could be used on any container where internal pressure measurements were needed without direct contact with the internal liquid. This may be particularly useful in the measurement of volatile or caustic liquids that would damage traditional diaphragm based pressure sensors.

### BRIEF DESCRIPTION OF DRAWINGS

The preferred embodiments of the invention will hereinafter be described in conjunction with the appended drawings provided to illustrate and not to limit the invention, where like designations denote like elements, and in which:

FIG. 1 presents a diagrammatic view of the device components positioned around a fluid-filled container.

FIG. 2 presents a diagrammatic view of the device components positioned around an axial cross-section view diagram of a lower leg and muscle compartments.

FIG. 3 presents a perspective side view of a cuff-style enclosure of the device applied around a leg to measure the compartment pressure in the leg.

### DETAILED DESCRIPTION

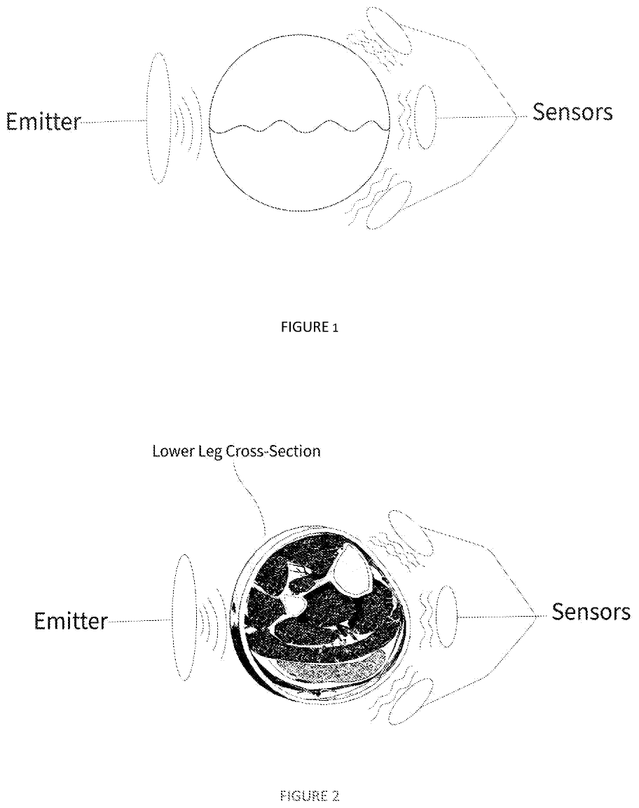

The following detailed description is merely exemplary in nature and is not intended to limit the described embodiments or the application and uses of the described embodiments. As used herein, the word “exemplary” or “illustrative” means “serving as an example, instance, or illustration.”. Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of the description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is directed to a device composed of one or more vibratory sound wave emitters and one or more microphone receivers (detectors) that measure when the various natural resonant frequencies of a target container or body part have been induced.

Referring initially to FIG. 1, a diagrammatic view of a fluid-filled container where a pressure measurement is desired without penetrating the container. Illustrated in accordance with an exemplary embodiment of the present invention is an emitter that generates a range of different frequency vibrations (sound waves) which then resonate the container. The receivers positioned around the container detect when resonant frequencies have been elicited in the container and these readings are transmitted to a central processor for calculation.

Referring to FIG. 2, a diagrammatic view of an axial, cross-section view, of a lower leg with internal muscular fascial compartments and bones, surrounded by the components of the present invention: an emitter that generates a range of different frequency vibrations (sound waves) which then cause the leg to resonate. One or more receivers are positioned around the leg to detect the induced natural resonant frequency points and send them to a central processor for calculation.

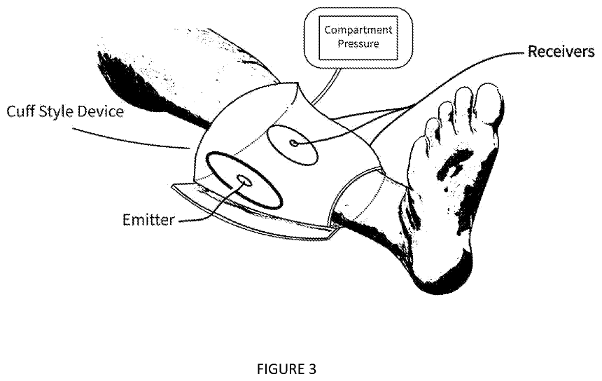

Referring to FIG. 3, a side perspective view of a leg in which compartment pressure measurement is desired. A cuff-style device enclosure that encases and contains the components, the emitters and receivers, is positioned around the leg. The emitter produces varying frequency wavelength vibrations (sound waves) to induce one or more resonant points in the leg. The receivers detect the increased amplitude indicating natural resonance points and send this information to the central processor for calculation of the compartment pressure which may then be displayed on a screen or similar device as shown, or transmitted to a monitoring health professional.

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

1. A method of measuring internal pressure of a muscle fascial compartment or other containers comprising:
Emitting a rage of varying frequency vibrations (sound waves) into the target compartment or container;
Identifying one or multiple natural resonant frequencies of the target compartment or container;
and
calculating the internal pressure by mathematical calculations with known data from previous measurements and experiments or measurements from the contralateral arm or leg.

2. A device for measuring muscle fascia compartment pressure or other containers, comprising;
one or more vibration (sound wave) emitters that direct a varying range of frequency and amplitude sound waves into the target body part or container to be measured to induce resonance at one or more natural resonant frequencies;
one or more receivers to measure the induced natural resonant frequencies;
and
a processor to calculate the internal pressure through use of mathematical calculations with previously obtained data values from experiments or the contralateral limb.
